(19) United States
(12) Patent Application Publication
(10) Pub. No.: US 2007/0207096 A1
(43) Pub. Date: Sep. 6, 2007

(54) Nail varnish having a gelled texture

(76) Inventors: Virginie Puisset, Paris (FR); Laurence Guerchet, Vitry Sur Seine (FR); Lizabeth-Anne Coffey-Dawe, Aulnay Sous Bois (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/705,503

(22) Filed: Feb. 13, 2007

Related U.S. Application Data
(60) Provisional application No. 60/777,579, filed on Mar. 1, 2006.

(30) Foreign Application Priority Data
Feb. 13, 2006 (FR) 06 50509

(51) Int. Cl. A61K 8/25; A61K 8/41

(52) U.S. Cl. 424/61

(57) ABSTRACT

Disclosed herein is a nail varnish comprising a cosmetically acceptable medium and at least one thixotropic thickener, wherein the composition has a viscosity at 25° C. of at least 0.6 Pa·s. Also disclosed herein is a cosmetic nail makeup process comprising lowering the viscosity of a nail varnish composition with a viscosity at 25° C. of at least 0.6 Pa·s, with a non-chemical action, for instance, a mechanical action, simultaneously with or prior to the application of the composition to the nails.

This application claims benefit of U.S. Provisional Application No. 60/777,579, filed Mar. 1, 2006, the contents of which are incorporated herein by reference. This application also claims benefit of priority under 35 U.S.C. § 119 to French Patent Application No. FR 06 50509, filed Feb. 13, 2006, the contents of which are also incorporated herein by reference.

Disclosed herein is a high-viscosity nail varnish having a gelled texture. Also disclosed herein is a process for coating the nails comprising applying a varnish of the present disclosure to the nails.

The colored or transparent nail varnish composition of the present disclosure may be used as a varnish base or “base coat”, as a nail makeup product, as a finishing composition, also known as a “top coat”, to be applied over the nail makeup product, and/or as a cosmetic nailcare product.

Conventional nail varnishes are in liquid or fluid form and are generally contained in bottles. They generally comprise solid particles such as pigments, nacres, and/or fillers, which are in dispersion in the continuous aqueous medium or the organic solvent medium of the composition..

This fluid form may provide good dispersion of the pigments so as to preserve the homogeneity of the color of the liquid varnish and also of the film of varnish once applied to the nails.

However, these particles have a tendency to sediment over time, due to their density, which is higher than that of the continuous medium in which they are dispersed. This sedimentation may result in a change in the microscopic appearance of the composition, for instance, in the case of colored nail varnishes, in heterogeneity of the color of the varnish.

It would thus be desirable to provide nail varnishes that have a good dispersion of the particles, for example, of the pigments, and thus good stability and good homogeneity of the color over time, and also a film that has satisfactory covering power when applied to the nails.

In addition, from a practical viewpoint, it would be advantageous to provide varnishes with novel textures that are easy to manipulate (e.g., no problems of spilling and/or dripping).

The present inventors have found that at least one of the advantages mentioned above can be obtained by using a high-viscosity nail varnish composition in non-liquid form and having a gelled texture, which may allow uniform dispersion of the pigments, wherein the composition exhibits a thixotropic behavior.

Furthermore, this gelled texture may allow for better organization and orientation of the coloring particles (for example, the nacres) in the composition when it is applied to the nails and then during the drying of the film of varnish, thus making it possible to obtain a color effect and gloss that may be superior to those of films derived from conventional fluid nail varnishes in which the coloring particles do not follow a preferential orientation.

In addition, contrary to conventional nail varnishes, this composition may exhibit improved flow properties, e.g. does not drip and makes it possible to obtain, after application to the nails, a film that dries quickly while at the same time being uniform and smooth; and may have good staying power and gloss properties.

Disclosed herein is a nail varnish composition comprising a cosmetically acceptable medium and at least one thixotropic thickener, wherein the composition has a viscosity at 25° C. of at least 0.6 Pa·s.

In one embodiment, the nature and/or amount of the at least one thickener are such that, in response to a non-chemical action, for example, a mechanical action, prior to or simultaneously with the application of the composition to the nails, the viscosity of the composition may be reversibly lowered to a value less than or equal to 0.4 Pa·s, for instance, a value less than or equal to 0.3 Pa·s.

Measurement of the Viscosity

The viscosity of the composition is measured at 25° C. using a Rheomat 180 viscometer (from the company Lamy) equipped with an MS-R1, MS-R2, MS-R3, MS-R4, or MS-R5 spindle chosen as a function of the consistency of the composition, rotating at a spin speed of 200 rpm. The measurement is taken after 10 minutes of rotation. The viscosity measurements are performed not more than one week after manufacture.

The nail varnish of the present disclosure may have a viscosity ranging from 0.6 to 20 Pa·s, for example, from 0.7 to 15 Pa·s, or from 0.75 to 10 Pa·s.

Disclosed herein is a nail varnish composition comprising a cosmetically acceptable medium and at least one thixotropic thickener, wherein the composition exhibits a thixotropic behavior.

Also disclosed herein is a nail varnish composition comprising a cosmetically acceptable medium and at least one thixotropic thickener, wherein the composition exhibits a rheofluidizing behavior.

Further disclosed herein is the use of a composition of the present disclosure to obtain a film of varnish that has good gloss properties and/or good covering power when applied to the nails.

Still further disclosed herein is the use of a composition of the present disclosure to obtain a film of varnish that dries quickly when applied to the nails.

Also disclosed herein is a cosmetic nail makeup process comprising lowering the viscosity of a nail varnish composition with a viscosity at 25° C. of at least 0.6 Pa·s, by means of a non-chemical action, for instance, a mechanical action, simultaneously with or prior to the application to the nails of the said composition.

Further disclosed herein is the use of a sufficient amount of at least one thixotropic thickener to obtain a stable nail varnish composition with a viscosity at 25° C. of at least 0.6 Pa·s.

As used herein, the term “cosmetically acceptable medium” means a non-toxic medium that may be applied to human keratin materials, such as the nails.

In at least one embodiment, the composition according to the present disclosure has a thixotropic nature.

As used herein, the terms “composition with a thixotropic nature or thixotropic behavior”, or “thixotropic composition”, mean a structured composition that fluidizes (for example, its viscosity decreases) when a non-chemical action is applied thereto, such as a mechanical action, and which recovers all or part of its initial viscosity after a sufficient standing time, which may be of varying lengths depending on the application, at room temperature.

In at least one embodiment, the composition may have at least one of the following properties:
the composition has a rheofluidizing character, i.e., the viscosity of the composition decreases when increasing shearings are applied to the composition;
the composition, after the application of shearing, fluidizes (for example, its viscosity decreases). At least one of the viscosity, the consistency, and the elasticity of the composition after its destructuration, for instance, after a rest time of one minute after having applied the shearing, is less than that of the composition before the application of the shearing;
the composition regenerates partially or completely its initial structure after a sufficient rest time and the restructuring of the composition is delayed in time. The restructuring of the composition therefore does not occur instantly but in a deferred manner over time. For instance, the composition, when subjected to a constant shearing of 1000 s⁻¹ for one minute, partly or completely returns to its initial viscosity after a sufficient rest time which may be of varying lengths depending on the application.

A definition of a thixotropic composition is indicated, for example, in the book “Comprendre la rhéologie—De la circulation du sang a la prise du beton” by P. Coussot and J. L. Grossiord, EDP Science, 2002, pages 16 and 17.

The thixotropic behavior of the composition may be evaluated by measuring the viscosity of the composition under low and high stresses, as described below.

The measurements were performed on a controlled-stress rheometer Haake RheoStress RS 600 from the company ThermoRhéo, equipped with a thermostatically maintained bath and a stainless-steel spindle of cone/plate geometry, with a diameter of 35 mm, an angle of 2°, and a gap of 0.104 mm. The two surfaces are striated to limit the sliding phenomena at the walls of the plates. An anti-evaporation device (solvent bell) is used.

The measurements are performed at 20° C.±1° C.

First, the sample is maintained at 20° C.±1 ° C. for 300 seconds (without shearing).

a) Increasing stresses are applied to the sample, starting from an initial stress equal to 0 Pa to arrive at a final stress equal to 500 Pa (which corresponds to a shearing going from 10⁻¹s⁻¹ to 400 s⁻¹), the stresses being applied only once.

The measurement is taken on 40 logarithmically distributed points.

Time is allowed for a stable value to be obtained between each stress, the waiting time between each stress being 30 s.

Then the graphical representation of the change in viscosity, noted as η, as a function of the shear rate, noted as a, is traced.

b) Next, the composition is destructured by applying a continuous shear rate \dot{γ} of 400 s⁻¹ (corresponding to a stress of 500 Pa) for 300 seconds.

c) Then decreasing stresses are applied to the sample, starting from an initial stress equal to 500 Pa to arrive at a final stress equal to 0 Pa (which corresponds to a shear rate going from 400 s⁻¹ to 10⁻³ s⁻¹), the stresses being applied only once. In, the range under consideration, the maximum value of 400 s⁻¹ must be taken into account with a measurement uncertainty of ±10 s⁻¹.

The measurement is taken on 40 logarithmically distributed points.

Time is allowed for a stable value to be obtained between each stress, the waiting time between each stress being 30 s.

Then the graphical representation of the change in viscosity, noted as A, as a function of the shear rate, noted as a, is traced.

d) Next, the composition is subjected to a stress of 10 Pa for 1000 s, and the change in viscosity as a function of the time is measured.

e) Increasing stresses are again applied to the sample, starting from an initial stress equal to 0 Pa to arrive at a final stress equal to 500 Pa,(which corresponds to a shear rate going from 10⁻² s⁻¹ to 300 s⁻¹), the stresses being applied only once.

Then the graphical representation of the change in viscosity, noted as a, as a function of the shear rate, noted as y, is traced.

The measurement is taken on 40 logarithmically distributed points.

Time is allowed for a stable value to be obtained between each stress, the waiting time between each stress being 30 s.

The results are analyzed by means of the graphical representation of the change in viscosity, noted as η, as a function of the shear rate, noted as \dot{γ}. The stress τ, the viscosity η and the shear rate \dot{γ} are linked by the following relationship:

$$γ = \frac{τ}{η} .$$

In at least one embodiment, the viscosity of the composition of the present disclosure, as measured at step e), at a shear rate of 4.10⁻² s⁻¹, ranges from 10² to 10⁴ Pa·s, for example, from 5.10² to 5.1 Pa·s, or from 600 to 4000 Pa·s.

The rheofluidizing character of the composition may, in at least one embodiment, be characterized by a difference (viscosity as measured at step c), at a shear rate of 100 s⁻¹—viscosity as measured at step e), at a shear rate of 4.10⁻² s⁻¹), ranging from 10 to 10⁵ Pa·s, for example, from 10² to 10⁴ Pa·s.

The thixotropic character of the composition may, in at least one embodiment, be characterized by a difference (viscosity as measured at step c), at a shear rate of 1 s⁻¹—viscosity as measured at step e), at a shear rate of 1 s⁻¹), greater than or equal to 1 Pa.s, for example, greater than or equal to 10 Pa·s, greater than or equal to 20 Pa·s, greater than or equal to 30 Pa·s, or greater than or equal to 40 Pa·s.

In another embodiment, the difference (viscosity as measured at step c), at a shear rate of 1 s⁻¹—viscosity as measured at step e), at a shear rate of 1 s⁻¹), may range from 1 to 1000 Pa·s, for instance, from 20 to 500 Pa·s, or from 40 to 200 Pa·s.

Measurement of the Viscoelastic Properties

In at least one embodiment, the compositions of the present disclosure may exhibit viscoelastic behavior, with a main elastic behavior.

In general, and as used herein, a material is said to be “viscoelastic” when, under the effect of shear, it has both the characteristics of an elastic material, i.e. capable of storing energy, and the characteristics of a viscous material, i.e. capable of dissipating energy.

The viscoelastic behavior of the compositions in accordance with the present disclosure may be characterized by their modulus of stiffness G*, the elasticity δ, and the yield point τ_{C}. These parameters are defined, for example, in the publication “Initiation à la rheologie [Introduction to Rheology]”, G. Couarraze and J. L. Grossiord, 2nd edition, 1991, published by Lavoisier-Tec 1 Doc.

The measurements were performed on an ( Haake RheoStress 600®>> controlled-stress rheometer from the company ThermoRh6o, equipped with a thermostatically maintained bath and a stainless-steel spindle of plate/plate geometry, the plate having a diameter of 20 mm and a gap (distance between the bottom plate—known as the stator plate—on which the composition is deposited and the top plate—known as the rotor plate) of 1 mm. The two plates are striated to limit the sliding phenomena at the walls of the plates. An anti-evaporation device (solvent bell) is used.

The measurements are performed at 20° C.±1 ° C.

The composition is subjected to a continuous stress under a stress τ(t) varying sinusoidally according to a pulsation ω(ω=2ΠN, N being the frequency of the shearing applied).

The stress τ(t) and the deformation γ(t) are defined respectively by the following relationships:
τ(t)=τ₀ cos ωt γ(t)=γ₀ cos(ωt−δ)
τ₀ being the maximum amplitude of the stress and γ₀ being the maximum amplitude of the deformation.

The measurements are carried out at a frequency of 1 Hz (N=1 Hz).

First, the sample is maintained at 20° C.±1° C. for 300 seconds (without shearing).

Increasing stresses are applied to the sample, starting from an initial stress equal to 0.01 Pa to arrive at a final stress equal to 1000 Pa, until destructuration of the sample, the stresses being applied only once.

The variation of the modulus of stiffness G* (corresponding to the ratio τₒ/γₒ.) and of the elasticity δ (corresponding to the dephasing angle of the applied stress relative to the measured strain) is thus measured as a function of the applied stress τ(t).

The strain of the composition for the stress zone in which the variation of the modulus of stiffness G and the elasticity δ is less than 7% (microstrain region) is measured, and the “plateau” parameters Gₚ and δₚ are thus determined.

The strain stress τ_{c} (the composition does not flow under its own weight, and the strain stress correspond to the minimal strength to be applied to the composition to make it flow) is determined from the graphic representation G*=f(τ).

It corresponds to the value of τ at the intersection of the two tangents to the curves G*=f(τ) for the low values of τ and for the high values of τ.

The viscoelastic behavior of the compositions according to the present disclosure may, in at least one embodiment, be characterized by a plateau modulus of stiffness Gp greater than 100 Pa, for example, greater than 500 Pa.

Thus, disclosed herein is a composition comprising a cosmetically acceptable medium and at least one thixotropic thickener, wherein the composition has a plateau modulus of stiffness Gp greater than 100 Pa.

In another embodiment, the composition according to the present disclosure exhibits a plateau modulus of stiffness Gp ranging from 100 to 2×10⁶ Pa·s, for instance, from 5×10² to 10⁴ Pars, such as from 800 to 4000 Pa·s, or from 1000 to 3000 Pa·s.

The composition according to the present disclosure may, in at least one embodiment, exhibit an elasticity δₚ ranging from 2° to 30°, for example, from 15° to 25° and a strain stress τ_{c} ranging from 10 Pa to 30000 Pa, for instance, from 30 Pa to 500 Pa, such as from 50 to 200 Pa.

Thixotropic Thickeners

The composition according to the present disclosure comprises at least one thixotropic thickener in an amount that is sufficient to give the composition a viscosity at rest sufficient to give it the desired texture and thixotropic behavior.

In at least one embodiment, the nature and/or amount of the at least one thickener is such that, in response to a non-chemical action, for instance, a mechanical action, prior to or simultaneously with the application of the composition to the nails, the viscosity of the composition may be reversibly lowered to a value less than or equal to 0.4 Pa·s, for example, less than or equal to 0.3 Pa·s.

The thixotropic thickener may be present in the composition in an amount greater than or equal to 1.7% by weight, for example, ranging from 1.7% to 15% by weight, or greater than or equal to 2% by weight, for example, ranging from 2% to 10% by weight, or ranging from 2% to 7.5% by weight, relative to the total weight of the composition.

The at least one thickener may be chosen, for example, from hydrophilic or organophilic clays, hydrophilic or hydrophobic fumed silicas, elastomeric organopolysiloxanes, and mixtures thereof.

Clays are silicates containing a cation that may be chosen from calcium, magnesium, aluminium, sodium, potassium, and lithium cations, and mixtures thereof. As used herein, the term “hydrophilic clay” means a clay that is capable of swelling in water; this clay swells in water and forms after hydration a colloidal dispersion.

Examples of such products include, but are not limited to, clays of the smectite family such as montmorillonites, hectorites, bentonites, beidellites, and saponites, clays of the vermiculite family, stevensite, and chlorites.

These clays may be of natural or synthetic origin.

Non-limiting examples of hydrophilic clays include smectites such as saponites, hectorites, montmorillonites, bentonites, beidellite and, in at least one embodiment, synthetic hectorites (also known as laponites), for instance, the products sold by the company Laporte under the names Laponite XLG, Laponite RD, and Laponite RDS (these products include, for example, sodium magnesium silicates and sodium lithium magnesium silicates); bentonites, for instance the product sold under the name Bentone HC by the company Rheox; magnesium aluminium silicates, which may be hydrated, for instance, the products sold by the company Vanderbilt Company under the names Veegum Ultra, Veegum HS, and Veegum DGT, and calcium silicates, such as the product in synthetic form sold by the company under the name Micro-cel C.

The organophilic clays are clays modified with chemical compounds that make the clay capable of swelling in solvent media.

The clay may be chosen, for example, from montmorillonite, bentonite, hectorite, attapulgite, sepiolite, and mixtures thereof. In one embodiment, the clay is chosen from bentonite and hectorite.

The chemical compound used to modify the organophilic clay may be chosen, for instance, from quaternary amines, tertiary amines, amine acetates, imidazolines, amine soaps, fatty sulfates, alkyl aryl sulfonates, amine oxides, and mixtures thereof.

Suitable organophilic clays include, but are not limited to, quaternium-18 bentonites such as those sold under the names Bentone 3, Bentone 38, and Bentone 38V by the company Elementis, Tixogel VP by the company United Catalyst, and Claytone 34, Claytone 40, and Claytone XL by the company Southern Clay; stearalkonium bentonites such as those sold under the names Bentone 27V by the company Elementis, Tixogel LG by the company United Catalyst, and Claytone AF and Claytone APA by the company Southern Clay; and quaternium-18/benzalkonium bentonites such as those sold under the names Claytone HT and Claytone PS by the company Southern Clay.

The hydrophilic fumed silicas may be obtained by high-temperature hydrolysis of a volatile silicon compound in an oxyhydric flame, producing a finely divided silica. Hydrophilic silicas have a large number of silanol groups at their surface. Such hydrophilic silicas are sold, for example, under the names Aerosil 130®, Aerosil 200®, Aerosil 255®, Aerosil 300®, and Aerosil 380® by the company Degussa, and Cab-O-Sil HS-5®, Cab-O-Sil EH-5®, Cab-O-Sil LM-130®, Cab-O-Sil MS-55®, and Cab-O-Sil M-5® by the company Cabot.

The hydrophobic fumed silicas may be obtained by modification of the surface of the silica via a chemical reaction that generates a reduction in the number of silanol groups, these groups possibly being substituted, for example, with hydrophobic groups.

The hydrophobic groups may be chosen, for instance, from:

trimethylsiloxyl groups, which may be obtained by treating fumed silica in the presence of hexamethyldisilazane. Silicas thus treated are known as “Silica silylate” according to the CTFA (6th edition, 1995). They are sold, for example, under the references Aerosil R812® by the company Degussa, and Cab-O-Sil TS-530® by the company Cabot,

dimethylsilyloxyl or polydimethylsiloxane groups, which may be obtained by treating fumed silica in the presence of polydimethylsiloxane or dimethyldichlorosilane. Silicas thus treated are known as “Silica dimethyl silylate” according to the CTFA (6th edition, 1995). They are sold, for example, under the references Aerosil R972® and Aerosil R974® by the company Degussa, and Cab-O-Sil TS-610® and Cab-O-Sil TS-720® by the company Cabot.

In at least one embodiment, the elastomeric polyorganosiloxanes may be partially or totally crosslinked and may possibly have a three-dimensional structure.

The elastomeric polyorganosiloxanes combined with a fatty phase may be in the form of a gel comprising at least one elastomeric polyorganosiloxane combined with a fatty phase, present in at least one hydrocarbon-based oil and/or at least one silicone oil. They may be chosen, for example, from the crosslinked polymers described in European Patent Application No.0 295 886.

According to one embodiment of the present disclosure, the elastomeric organopolysiloxanes may be obtained by addition reaction and crosslinking of:

(a) at least one organopolysiloxane comprising at least two lower alkenyl groups per molecule;

(b) at least one organopolysiloxane comprising at least two hydrogen atoms linked to a silicon atom per molecule; and

(c) a platinum-type catalyst.

The elastomeric organopolysiloxanes combined with a fatty phase may also be chosen from those described in U.S. Pat. No. 5,266,321, for instance, polyorganopoly-siloxanes comprising units R₂SiO and RSiO_{1.5} and possibly units R₃SiO_{0.5} and/or SiO₂ in which the radicals R, which may be identical or different, are chosen from hydrogen, alkyl groups such as methyl, ethyl, and propyl, aryl groups such as phenyl and tolyl, and unsaturated aliphatic groups such as vinyl, and in which the weight ratio of the units R₂SiO to the units RSiO_{1.5} ranges from 1/1 to 30/1.

According to one embodiment, the at least one thixotropic thickener is chosen from organophilic modified clays such as hectorite modified with benzyldimethylammonium stearate.

Additional Thickeners

The composition according to the present disclosure may also comprise at least one additional thickener other than the thixotropic thickeners described above.

This at least one additional thickener is not capable by itself of giving the composition the thixotropic nature (i.e., it is a non-thixotropic thickener); but may make it possible to adjust the viscosity of the composition to obtain uniform flow.

The at least one additional thickener may be chosen, according to the cosmetically acceptable medium of the composition, for example, from:

hydrophilic thickeners such as
guar gum, quaternized guar gum, nonionic guar gums, xanthan gum, carob gum, scleroglucan gum, gellan gum, rhamsan gum, karaya gum, alginates, maltodextrin, starch and derivatives thereof, and hyaluronic acid and salts thereof,
polyglyceryl (meth)acrylate polymers,
polyvinylpyrrolidone,
polyvinyl alcohol,
crosslinked acrylamide polymers and copolymers, and
associative polymers, such as associative polyurethanes,

organophilic thickeners, for instance:
alkyl guar gums (with a C₁-C₆ alkyl group), such as those described in European Patent Application No. 0 708 114;
oil-gelling polymers, for instance, triblock or star polymers resulting from the polymerization or copolymerization of at least one monomer comprising an ethylenic group, for instance the polymers sold under the name Kraton; and
polyamides resins comprising alkyl groups comprising from 12 to 22 carbon atoms, such as those described in U.S. Pat. No. 5,783,657; and

hydrophobic thickeners such as polysaccharide alkyl ethers (for instance, those in which the alkyl group comprises from 1 to 24, for example, from 1 to 10, from 1 to 6, or from 1 to 3 carbon atoms), such as those described in European Patent Application No. 0 898 958.

The at least one additional thickener may be present in the composition in an amount ranging from 0.1% to 20% by weight, for example, from 0.1% to 10% by weight relative to the total weight of the composition.

Organic Solvent Medium

The composition according to the present disclosure may comprise an organic solvent medium comprising at least one organic solvent chosen from:

ketones that are liquid at room temperature, such as methyl ethyl ketone, methyl isobutyl ketone, diisobutyl ketone, isophorone, cyclohexanone, and acetone;

alcohols that are liquid at room temperature, such as ethanol, isopropanol, diacetone alcohol, 2-butoxyethanol, and cyclohexanol;

propylene glycol ethers that are liquid at room temperature, such as propylene glycol monoethyl ether, propylene glycol monoethyl ether acetate, and dipropylene glycol n-butyl ether;

cyclic ethers such as y-butyrolactone;

short-chain esters (comprising from 3 to 8 carbon atoms in total), such as ethyl acetate, butyl acetate, methyl acetate, propyl acetate, isopropyl acetate, isopentyl acetate, methoxypropyl acetate, and butyl lactate;

ethers that are liquid at room temperature, such as diethyl ether, dimethyl ether, and dichlorodiethyl ether;

alkanes that are liquid at room temperature, such as decane, heptane, dodecane, and cyclohexane;

alkyl sulfoxides such as dimethyl sulfoxide;

aldehydes that are liquid at room temperature, such as benzaldehyde and acetaldehyde;

ethyl 3-ethoxypropionate;

carbonates such as propylene carbonate and dimethyl carbonate;

acetals such as methylal;

and mixtures thereof.

The organic solvent medium may me present in the composition in an amount ranging from 30% to 97% by weight, for example, from 50% to 95% by weight relative to the total weight of the composition.

The composition according to the present disclosure may comprise an aqueous medium.

The aqueous medium may be present in the composition in an amount ranging from 5% to 95% by weight, for instance, from 50% to 70% by weight relative to the total weight of the composition.

Film-Forming Polymers

The composition may further comprises at least one film-forming polymer.

As used herein, the term “film-forming polymer” means a polymer that is capable, by itself or in the presence of an auxiliary film-forming agent, of forming a continuous film that adheres to a support such as keratin materials.

Examples of film-forming polymers that may be used in the composition of the present disclosure include, but are not limited to, free-radical synthetic polymers, polycondensate synthetic polymers, polymers of natural origin, and mixtures thereof.

In at least one embodiment, the film-forming polymer may be chosen from cellulose-based polymers such as nitrocellulose, cellulose acetate, cellulose acetobutyrate, cellulose acetopropionate, and ethylcellulose; polyurethanes; acrylic polymers; vinyl polymers; polyvinyl butyrals; alkyd resins; and resins derived from aldehyde condensation products such as arylsulfonamide-formaldehyde resins, for instance toluenesulfonamide-formaldehyde resin, arylsulfonamide-epoxy resins, and ethyltosylamide resins.

Examples of commercially available film-forming polymers that may be used include, but are not limited to, nitrocellulose RS 1/8 sec.; RS 1/4 sec.; RS 1/2 sec.; RS 5 sec.; RS 15 sec.; RS 35 sec.; RS 75 sec.; RS 150 sec.; AS 1/4 sec.; AS 1/2 sec.; SS 1/4 sec.; SS 1/2 sec.; SS 5 sec.; sold, for example, by the company Hercules; the toluenesulfonamide-formaldehyde resins Ketjenflex MS80 from the company Akzo, Santolite MHP and Santolite MS 80 from the company Faconnier, and Resimpol 80 from the company Pan Americana, the alkyd resin Beckosol ODE 230-70-E from the company Dainippon, the acrylic resin Acryloid B66 from the company Rohm & Haas, and the polyurethane resin Trixene PR 4127 from the company Baxenden.

According to one embodiment of the present disclosure, the at least one film-forming polymer is a film-forming linear block ethylenic polymer, for instance, a linear block ethylenic polyer which comprises at least one first block and at least one second block with different glass transition temperatures (Tg), the said first and second blocks being linked together by an intermediate block comprising at least one constituent monomer of the first block and at least one constituent monomer of the second block.

In another embodiment, the first and second blocks of the block polymer are mutually incompatible.

Such polymers are described, for example, in European Patent No.1 411 069 and International Patent Application Publication No. WO 04/028 488.

The at least one film-forming polymer may be present in the composition in a dry matter content ranging from 0.1% to 60% by weight, for example, from 2% to 40% by weight, or from 5% to 25% by weight relative to the total weight of the composition.

Auxiliary Film-Forming Agents

To improve the film-forming properties of the nail varnish composition, the composition may also comprise at least one auxiliary film-forming agent.

Such an auxiliary film-forming agent may be chosen from any compound known to those skilled in the art as being capable of satisfying the desired function, for example, plasticizers and coalescers for the at least one film-forming polymer.

Thus, in at least one embodiment, the composition may also comprise at least one plasticizer and/or at least one coalescer. Examples of suitable plasticizers and coalescers include, but are not limited to:

glycols and derivatives thereof, such as diethylene glycol ethyl ether, diethylene glycol methyl ether, diethylene glycol butyl ether, diethylene glycol hexyl ether, ethylene glycol ethyl ether, ethylene glycol butyl ether, and ethylene glycol hexyl ether;

glycol esters;

propylene glycol derivatives such as propylene glycol phenyl ether, propylene glycol diacetate, dipropylene glycol ethyl ether, tripropylene glycol methyl ether, diethylene glycol methyl ether, and propylene glycol butyl ether;

acid esters, for instance, carboxylic acid esters, such as citrates, phthalates, adipates, carbonates, tartrates, phosphates, and sebacates;

oxyethylenated derivatives, such as oxyethylenated oils, for example, plant oils such as castor oil; and

mixtures thereof.

The type and amount of the at least one auxiliary film-forming agent, such as plasticizers and/or coalescers may be chosen by a person skilled in the art on the basis of his general knowledge.

For example, the at least one auxiliary film-forming agent may be present in the composition in an amount ranging from 0.01% to 20%, such as from 0.5% to 10% by weight relative to the total weight of the composition.

Spreading Agents

According to one embodiment, the composition according to the invention may comprise at least one spreading agent to promote the application of the composition to the nails. It may be chosen from linear or cyclic silicone oils, for instance, those with a viscosity ≦6 centistokes (6×10⁻⁶ m²/s) and comprising, for example, from 3 to 6 silicon atoms, these silicones optionally comprising at least one group chosen from alkyl and alkoxy groups comprising 1 or 2 carbon atoms. Silicone oils that are suitable for use in accordance with the present disclosure include, for instance, octamethylcyclotetrasiloxane, decamethylcyclopentasiloxane, dodecamethylcyclohexasiloxane, heptamethylhexyl-trisiloxane, heptamethyloctyltrisiloxane, hexamethyldisiloxane, octamethyltrisiloxane, decamethyltetrasiloxane, dodecamethylpentasiloxane, and mixtures thereof.

The at least one spreading agent may be present in the composition in an amount ranging from 0.1% to 15% by weight, for instance, from 0.5% to 10% by weight, or from 1 % to 5% by weight relative to the total weight of the composition.

Dyestuffs

The composition according to the invention may also comprise at least one dyestuff chosen from water-soluble dyes and pulverulent dyestuffs, for instance, pigments, nacres, and flakes that are known to those skilled in the art. The dyestuffs may be present in the composition in an amount ranging from 0.01 % to 60% by weight, for example, from 1% to 40% by weight relative to the weight of the composition.

As used herein, the term “pigments” means white or colored, mineral or organic particles of any form, which are insoluble in the physiological medium and are intended to color the composition.

As used herein, the term “nacres” means iridescent particles of any form, for instance, those produced by certain molluscs in their shell and synthesized particles.

The pigments may be white or colored, and mineral and/or organic. Examples of suitable mineral pigments include, but are not limited to, titanium dioxide, optionally surface-treated, zirconium oxide, cerium oxide, zinc oxide, iron (e.g., black, yellow, and red) oxide, chromium oxide, manganese violet, ultramarine blue, chromium hydrate, ferric blue, and metal powders, for instance, aluminium powder and copper powder.

Non-limiting examples of organic pigments include carbon black, pigments of D&C type, and lakes based on cochineal carmine, barium, strontium, calcium, and/or aluminium.

The nacreous pigments may be chosen, for instance, from white nacreous pigments such as mica coated with titanium or with bismuth oxychloride, colored nacreous pigments such as titanium mica coated with iron oxides, titanium mica coated with ferric blue or with chromium oxide, titanium mica coated with an organic pigment of the abovementioned type, and nacreous pigments based on bismuth oxychloride.

The water-soluble dyes include, for example, beetroot juice and methylene blue.

Fillers

The composition of the present disclosure may also comprise at least one filler, for instance, in an amount ranging from 0.01% to 50% by weight, such as from 0.01% to 30% by weight relative to the total weight of the composition. As used herein, the term “fillers” means colorless or white, mineral or synthetic particles of any form, which are insoluble in the medium of the composition irrespective of the temperature at which the composition is manufactured. These fillers serve, for example, to modify the rheology and/or the texture of the composition.

The at least one filler may be mineral or organic and of any form, for instance, platelet-shaped, spherical, and oblong, irrespective of the crystallographic form (for example lamellar, cubic, hexagonal, orthorhombic, etc.). Examples of suitable fillers include, but are not limited to, talc, mica, silica, kaolin, polyamide (Nylon®) powder (Orgasol® from Atochem), poly-β-alanine powder and polyethylene powder, tetrafluoroethylene polymer (Teflon®) powder, lauroyllysine, starch, boron nitride, hollow polymer microspheres such as polyvinylidene chloride/acrylonitrile microspheres, for instance Expancel® (Nobel Industrie), acrylic acid copolymer microspheres (Polytrap® from the company Dow Corning), and silicone resin microbeads (for example Tospearls® from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids comprising from 8 to 22 carbon atoms, for instance, from 12 to 18 carbon atoms, for example zinc stearate, magnesium stearate, lithium stearate, zinc laurate, and magnesium myristate.

Other Additives

The composition may also comprise at least one other ingredient commonly used in cosmetic compositions. Such ingredients may be chosen, for example, from spreading agents, wetting agents, dispersants, antifoams, preserving agents, UV-screening agents, active agents, surfactants, moisturizers, fragrances, neutralizers, stabilizers, and antioxidants.

It is understood that a person skilled in the art will take care to select the at least one optional additional compound, and/or the amount thereof, such that the advantageous properties of the composition are not, or are not substantially, adversely affected by the envisaged addition.

Process

Also disclosed herein is a cosmetic nail makeup process comprising lowering the viscosity of a nail varnish composition with a viscosity at 25° C. of at least 0.6 Pa·s, by means of a non-chemical action, simultaneously with or prior to the application of the composition to the nails.

According to one embodiment, the process comprises applying to the thixotropic composition, in the form of a gel conditioned in a container, a non-chemical action, for instance, a mechanical action, so as to fluidize and reduce the viscosity of the composition and to allow it to be applied to the nails. When the applied action ceases, the composition in its conditioning, after a rest time, regains its initial gel texture.

According to another embodiment, the process comprises taking up a sample of the thixotropic composition in its conditioning, and applying to the sample the non-chemical action so as to fluidize the composition simultaneously with its application to the nails.

The non-chemical action may be chosen from thermal actions, for instance a source of heat, mechanical actions such as an object via which a mechanical stress or shear is applied to the composition, and combinations thereof.

In at least one embodiment, the object via which mechanical stress or shear is applied may be an applicator, such as fine brushes, spatulas, and tips.

In another embodiment, the non-chemical action is a mechanical action.

The nail varnish composition of the present disclosure may be conditioned in a container delimiting at least one compartment, the container being closed by means of a closing member.

The container may be in any suitable form and may be at least partly made of a material such as glass. However, materials other than glass may be used, for instance thermoplastics such as PP and PE, and metals.

The closing member may be coupled to the compartment by screwing the container in the closed position. Alternatively, the coupling between the closing member and the container may take place other than by screwing, for instance, by click-fastening.

The container may be combined with an applicator that may be in the form of a fine brush comprising at least one tuft of bristles. Alternatively, the applicator may be in a form other than a fine brush, for example, chosen from spatulas and foam tips.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below. Unless otherwise mentioned, the amounts are given as weight percentages relative to the total weight of the composition.

### EXAMPLES

### Example 1

### Colored Nail Varnish

A nail varnish having the following composition (weight %) was prepared:

| | Example 1 |
|---|---|
| Nitrocellulose containing 30% isopropyl alcohol (viscosity: | 5.55 |
| E22-1/2 S) | |
| Nitrocellulose containing 30% isopropyl alcohol (Idyl E27 | 12.12 |
| from Bergerac) | |
| Nitrocellulose containing 30% isopropyl alcohol (Azur E80 | 0.08 |
| from Bergerac) | |
| Glycerophthalic alkyd resin esterified with branched fatty | 15.50 |
| acids, at 70% in ethyl acetate (Beckosol ODE 230 70E from | |
| Dainippon Ink and Chemicals) | |
| Isopropyl alcohol | 1.14 |
| Cyclopentadimethylsiloxane (DC245 Fluid from Dow | 2 |
| Corning) | |
| Polydimethylsiloxane 5 cSt (DC200 Fluid from Dow | 0.44 |
| Corning) | |
| Stearylbenzyldimethylammonium-modified hectorite | 2.56 |
| (Bentone 27 V from Elementis) | |
| Hydrophilic fumed silica (Aerosil 200 from Degussa) | 0.46 |
| Red 7 lake | 0.02 |
| Titanium oxide mica (Timiron Super Silk MP 1005 from | 0.55 |
| Merck) | |
| Titanium oxide mica (Flamenco Red 420 C from Engelhard) | 0.2 |
| Bismuth oxychloride | 0.78 |
| Ethyl acetate | 19.27 |
| Acetyl tributyl citrate | 7.54 |
| Butyl acetate | qs 100 |
| Citric acid monohydrate | 0.1 |

The composition had a viscosity at 25° C. of 0.820 Pars and was conditioned in a jar.

Mechanical agitation was applied to the composition using an applicator. The composition fluidized and the fluidized composition was then applied to the nails using the applicator. A glossy film that covered the nails well was obtained.

After a few seconds, the nail varnish regained its initial texture (viscosity close to the initial viscosity).

### Example 2

### Nail Varnish

| | Example 2 |
|---|---|
| Nitrocellulose containing 30% isopropyl alcohol (viscosity: | 5.2 |
| E22-1/2 S) | |
| Nitrocellulose containing 30% isopropyl alcohol (Idyl E27 | 13.70 |
| from Bergerac) | |
| Glycerophthalic alkyd resin esterified with branched fatty | 16.19 |
| acids, at 70% in ethyl acetate (Beckosol ODE 230 70E from | |
| Dainippon Ink and Chemicals) | |
| Isopropyl alcohol | 0.99 |
| Polydimethylsiloxane 5 cSt (DC200 Fluid from Dow | 0.5 |
| Corning) | |
| Stearylbenzyldimethylammonium-modified hectorite | 2.8 |
| (Bentone 27 V from Elementis) | |
| Hydrophilic fumed silica (Aerosil 200 from Degussa) | 0.526 |
| Ethyl acetate | 19.94 |
| Acetyl tributyl citrate | 7.96 |
| Butyl acetate | qs 100 |
| Citric acid monohydrate | 0.1 |

### Example 3

### Nail varnish

a) Preparation of a Pentaerythrityl Benzoate/Isophtalate/Isostearate Polycondensat

227,5 g of benzoic acid, 72,8 g of isostearic acid, and 118,3 g of pentaerythritol were introduced into a reactor, provided with mechanical stirring, argon inlet and distilling system and the temperature was increased to 110-130° C. under argon current, to obtain an homogeneous solution.

Then the temperature was progressively increased to about 180° C. and maintained for 2 hours. The temperature was increased to 220° C. and maintained until the acid number was lower or equal to 1, which takes about 18 hours.

The mixture was cooled at a temperature ranging from 100 to 130° C., then 91 g of isophtalic acid were introduced and the temperature was slowly increased to 220° C. over about 11 hours.

430 g of pentaerythrityl benzoate/isophta-late/isostearate polycondensate, under the form of a thick oil that solidified at room temperature, were obtained.

The polycondensate presented the following characteristics:

acid number=12.7

hydrdxyl number=49

η_{110° C.}=25.4 Poises (2540 mPa·s)

ratio between the aromatic monocarboxylic acid mole number and the non aromatic monocarboxylic acid mole number: 7.28.

420 g of the above polycondensate were brought to 100-120° C. and 180 g of butyl acetate was slowly added under stirring, then the whole was clarified by filtration on a sintered glass filter n°2.

After cooling at room temperature, 600 g of a polycondensate solution with 70% of polycondensate active material in ethyl acetate were obtained, under the form of a pale yellow viscous liquid having a viscosity at 25° C. of about 800 centipoises (mPa·s). b) the following nail varnish was prepared:

| | |
|---|---|
| Nitrocellulose containing 30% isopropyl alcohol (viscosity: | 4.84 |
| E22-1/2 S) | |
| Nitrocellulose containing 30% isopropyl alcohol (Idyl E27 | 12 |
| from Bergerac) | |
| Nitrocellulose containing 30% isopropyl alcohol (Azur E80 | 0.08 |
| from Bergerac) | |
| Glycerophthalic alkyd resin esterified with branched fatty | 2.45 |
| acids, at 70% in ethyl acetate (Beckosol ODE 230 70E | |
| from Dainippon Ink and Chemicals) | |
| Solution with 70% polycondensate active material in ethyl | 11.49 |
| acetate, as prepared at a) | |
| Isopropyl alcohol | 1.4 |
| Cyclopentadimethylsiloxane (DC245 Fluid from Dow | 2 |
| Corning) | |
| Polydimethylsiloxane 5 cSt (DC200 Fluid from Dow | 0.44 |
| Corning) | |
| Stearylbenzyldimethylammonium-modified hectorite | 3.19 |
| (Bentone 27 V from Elementis) | |
| Hydrophilic fumed silica (Aerosil 200 from Degussa) | 0.37 |
| Red 7 lake | 0.02 |
| Titanium oxide mica (Timiron Super Silk MP 1005 from | 0.55 |
| Merck | |
| Titanium oxide mica (Flamenco Red 420 C from | 0.2 |
| Engelhard) | |
| Bismuth oxychloride | 0.78 |
| Ethyl acetate | 15 |
| Acetyl tributyl citrate | 7.37 |
| Butyl acetate | Qsp 100 |
| Citric acid monohydrate | 0.13 |

The composition had a viscosity at 25° C., measured with a Rheomat 180, of 0.820 Pa·s

Mechanical agitation was applied to the composition using an applicator. The composition fluidized and the fluidized composition was then applied to the nails using the applicator. A glossy film that covered the nails well was obtained.

After a few seconds, the nail varnish regained its initial texture (viscosity close to the initial viscosity).

The thixotropic behavior of the composition was evaluated by measurement of the viscosity according to the protocol previously described.

The composition had a viscosity, as measured at step e) of 45 Pa·s.

The composition had a thixotropic behavior characterized by a viscosity difference: (viscosity as measured at step c), at a shear rate of 1 s⁻¹—viscosity as measured at step e), at a shear rate of 1 s⁻¹) of about 100 Pa·s.

The composition had a plateau modulus of stiffness Gp ranging from 1000 to 3000 Pa, of about 2000 Pa, an elasticity δₚ of 200, and a strain stress τ_{c} of 100 Pa.

What is claimed is:

1. A nail varnish composition comprising
a cosmetically acceptable medium and
at least one thixotropic thickener,
wherein the composition has a viscosity at 25° C. of at least 0.6 Pa·s.

2. The composition of claim 1, having a viscosity ranging from 0.6 to 20 Pa·s.

3. The composition of claim 2, having a viscosity ranging from 0.75 to 10 Pa·s.

4. A composition comprising
a cosmetically acceptable medium and
at least one thixotropic thickener,
wherein the composition has a thixotropic behavior such that when the composition is subjected to:
(a) increasing stress from 0 to 500 Pa·s (shear rate of 10⁻¹ s⁻¹ to 400 s⁻¹),
(b) a continuous stress of 500 Pa (shear rate of 400 s⁻¹) for 300 seconds,
(c) decreasing stress from from 500 to 0 Pa·s (shear rate of 400 s⁻±s⁻¹ to 10⁻¹s⁻¹),
(d) a continuous stress of 10 Pa·s for 1000 seconds, and then
(e) decreasing stress from from 500 to 0 Pa·s (shear rate of 400 s⁻¹±s⁻¹ to 10⁻¹s⁻¹), the viscosity at a shear rate of 1 s⁻¹ for a composition having been subjected to (a), (b) and
(c) minus the viscosity at a shear rate of 1 s⁻¹ for a composition having been subjected to all of (a) through (e) is greater than or equal to 1 Pa.s.

5. The composition of claim 4, wherein the difference of viscosities is greater than or equal to 10 Pa·s.

6. The composition of claim 5, wherein the difference of viscosities is greater than or equal to 40 Pa·s.

7. The composition of claim 4, wherein the difference of viscosities ranges from 1 to 1000 Pa·s.

8. The composition of claim 7, wherein the difference of viscosities ranges from 40 to 200 Pa·s.

9. A composition comprising
a cosmetically acceptable medium and
at least one thixotropic thickener,
wherein the composition has a plateau modulus of stiffness Gp greater than 100 Pa.

10. The composition of claim 9, wherein the plateau modulus of stiffness Gp ranges from 100 to 2×10⁶ Pa·s.

11. The composition of claim 10, wherein the plateau modulus of stiffness Gp ranges from 1000 to 3000 Pa·s.

12. The composition of claim 1, wherein the at least one thixotropic thickener is chosen from hydrophilic clays, organophilic clays, hydrophilic fumed silicas, hydrophobic fumed silicas, elastomeric organopolysiloxanes, and mixtures thereof.

13. The composition of claim 1, wherein the at least one thixotropic thickener is chosen from organophilic modified clays.

14. The composition of claim 13, wherein the at least one thixotropic thickener is hectorite modified with benzyldimethylammonium stearate.

15. The composition of claim 1, wherein the at least one thixotropic thickener further comprises at least one hydrophobic fumed silica.

16. The composition of claim 1, wherein the at least one thixotropic thickener is present in the composition in an amount ranging from 1.7% to 15% by weight relative to the total weight of the composition.

17. The composition of claim 16, wherein the at least one thixotropic thickener is present in the composition in an amount ranging from 2% to 7.5% by weight relative to the total weight of the composition.

18. The composition of claim 1, further comprising at least one film-forming polymer.

19. The composition of claim 18, wherein the at least one film-forming polymer is present in the composition in an amount ranging from 0.1% to 60% by weight relative to the total weight of the composition..

20. The composition of claim 19, wherein the at least one film-forming polymer is present in the composition in an amount ranging from 5% to 25% by weight relative to the total weight of the composition.

21. The composition of claim 1, further comprising at least one organic solvent.

22. The composition of claim 21, wherein the at least one organic solvent is present in the composition in an amount ranging from 30% to 97% by weight relative to the total weight of the composition.

23. The composition of claim 22, wherein the at least one organic solvent is present in the composition in an amount ranging from 50% to 95% by weight relative to the total weight of the composition.

24. The composition of claim 1, further comprising at least one spreading agent chosen from linear and cyclic silicone oils.

25. The composition of claim 24, wherein the at least one spreading agent has a viscosity of less than or equal to 6 centistokes (6×10⁻⁶ m²/s).

26. The composition of claim 24, wherein the at least one spreading agent is present in the composition in an amount ranging from 0.1% to 15% by weight relative to the total weight of the composition.

27. The composition of claim 26, wherein the at least one spreading agent is present in the composition in an amount ranging from 1% to 5% by weight relative to the total weight of the composition.

28. The composition of claim 1, further comprising at least one dyestuff chosen from water-soluble dyes, pigments, nacres, flakes, and mixtures thereof.

29. The composition of claim 28, wherein the at least one dyestuff is present in the composition in an amount ranging from 0.01% to 60% by weight relative to the weight of the composition.

30. The composition of claim 29, wherein the at least one dyestuff is present in the composition in an amount ranging from 1% to 40% by weight relative to the weight of the composition.

31. A method for providing the nails with a film of varnish that has desired gloss properties, desired covering power, and/or dries quickly when applied to the nails, comprising applying to the nails a composition comprising a cosmetically acceptable medium and at least one thixotropic thickener, wherein the composition has a viscosity at 25° C. of at least 0.6 Pa·s.

32. A cosmetic nail makeup process comprising lowering the viscosity of a nail varnish composition with a viscosity at 25° C. of at least 0.6 Pa·s, wherein said viscosity is lowered with a non-chemical action, simultaneously with or prior to the application of the composition to the nails.

33. The process of claim 32, wherein the non-chemical action is chosen from thermal and mechanical actions, and combinations thereof.

34. The process of claim 33, wherein the non-chemical action is a mechanical action.

35. The process of claim 34, wherein the mechanical action is applied via an applicator.

36. The process of claim 32, wherein the varnish has a viscosity ranging from 0.6 to 20 Pa·s.

37. The process of claim 36, wherein the varnish has a viscosity ranging from 0.75 to 10 Pa·s.

38. The process of claim 32, wherein the varnish composition comprises at least one thixotropic thickener.

39. The process of claim 38, wherein the at least one thixotropic thickener is chosen from hydrophilic clays, organophilic clays, hydrophilic fumed silicas, hydrophobic fumed silicas, elastomeric organopolysiloxanes, and mixtures thereof.

40. The process of claim 38, wherein the at least one thixotropic thickener is chosen from organophilic modified clays.

41. The process of claim 40, wherein the at least one thixotropic thickener is hectorite modified with benzyldimethylammonium stearate.

42. The process of claim 38, wherein the at least one thixotropic thickener further comprises at least one hydrophobic fumed silica.

43. The process of claim 38, wherein the at least one thixotropic thickener is present in the composition in an amount ranging from 1.7% to 15% by weight relative to the total weight of the composition.

44. The process of claim 43, wherein the at least one thixotropic thickener is present in the composition in an amount ranging from 2% to 7.5% by weight relative to the total weight of the composition.
